(19) United States
(12) Patent Application Publication
(10) Pub. No.: US 2026/0000468 A1
(43) Pub. Date: Jan. 1, 2026

(54) COMPUTER-ASSISTED CONTENT CONTROL IN DUAL OPERATOR CONFIGURATION

(71) Applicant: Intuitive Surgical Operations, Inc., Sunnyvale, CA (US)

(72) Inventors: Pourya SHIRAZIAN, Menlo Park, CA (US); Daniel PROKSCH, San Jose, CA (US)

(21) Appl. No.: 19/245,986

(22) Filed: Jun. 23, 2025

Related U.S. Application Data
(60) Provisional application No. 63/664,342, filed on Jun. 26, 2024.

(51) Int. Cl. A61B 34/00; A61B 34/35; G06F 3/04812; G06F 3/0482

(52) U.S. Cl. CPC A61B 34/25; A61B 34/35; G06F 3/04812; G06F 3/0482

(57) ABSTRACT

The present disclosure describes a system that allows control from two different consoles of a surgical robot. The system includes the surgical robot, a first console, a second console, and a computer system including a memory and a controller communicatively coupled to the memory. The controller of the system communicates, to the first console and the second console, video of a surgical site for the surgical robot based on determining that both the first console and the second console are operating in a first mode. The controller of the system, based on determining that the first console transitioned from operating in the first mode to operating in a second mode while the second console operates in the first mode, communicates, to the first console, video of a digital menu of applications while continuing to communicate video of the surgical site to the second console.

### RELATED APPLICATION

This application claims the benefit of U.S. provisional patent application Ser. No. 63/664,342, filed Jun. 26, 2024, which is hereby incorporated herein by reference.

### TECHNICAL FIELD

The present disclosure relates generally to medical systems. Specifically, the present disclosure relates to using a computer system that allows control from two different consoles of a robotic surgical system.

### BACKGROUND

Doctors use computer assisted medical systems to perform different medical tasks. For example, doctors may use computer assisted surgical systems to perform operations on patients, even remotely. These surgical systems include consoles that provide the doctors various views of surgical sites during the operations. The consoles may also include controls that the surgeons use to remotely operate robots that perform surgical operations on patients. Existing medical systems, however, include a single console, which limits usability and effectiveness in certain scenarios. For example, with a single console, collaboration between multiple surgeons during a procedure may be limited. These limits on collaboration may be a drawback in complex surgeries where the surgeons need to work together to successfully perform the operation. Moreover, with a single console, a surgeon's cognitive load may be increased. For example, the surgeon operating the single console may manage all or nearly all aspects of the procedure, including controlling the robotic arms, interpreting feedback from the imaging devices, and making decisions in real-time. This increased cognitive load may lead to fatigue and errors, which may negatively affect outcomes and patient health.

### SUMMARY

The present disclosure describes a system and method for allowing control from two different consoles of a robotic surgical system. According to an embodiment, a system includes a surgical robot, a first console, a second console, and a computer system including a memory and a controller communicatively coupled to the memory. The controller of the system communicates, to the first console and the second console, video of a surgical site for the surgical robot based on determining that both the first console and the second console are operating in a first mode. The controller of the system, based on determining that the first console transitioned from operating in the first mode to operating in a second mode while the second console operates in the first mode, further communicates, to the first console, video of a digital menu of applications while continuing to communicate video of the surgical site to the second console.

According to another embodiment, a method includes communicating, to a first console and a second console, video of a surgical site for a surgical robot based on determining that both the first console and the second console are operating in a first mode, and, based on determining that the first console transitioned from operating in the first mode to operating in a second mode while the second console operates in the first mode, communicate, to the first console, video of a digital menu of applications while continuing to communicate video of the surgical site to the second console.

The foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1A illustrates an example medical system.

FIGS. 1B and 1C illustrate example components in the system of FIG. 1A.

FIG. 2A illustrates an example medical system.

FIGS. 2B and 2C illustrate an example medical instrument system in the system of FIG. 2A.

FIG. 3 illustrates an example operation performed by the system of FIG. 1.

FIGS. 4A and 4B illustrate example operations performed by the system of FIG. 1.

FIG. 5 illustrates an example operation performed by the system of FIG. 1.

FIGS. 6A through 6C illustrate example operations performed by the system of FIG. 1.

FIGS. 7A through 7C illustrate example operations performed by the system of FIG. 1.

FIGS. 8A and 8B illustrate example operations performed by the system of FIG. 1.

FIG. 9 illustrates example operations performed by the system of FIG. 1.

FIGS. 10A through 10C illustrate example operations performed by the system of FIG. 1.

FIG. 11 is a flowchart of an example method performed by the system of FIG. 1.

FIGS. 12A and 12B illustrate example operations performed by the system of FIG. 1.

### DETAILED DESCRIPTION

The present disclosure is generally directed to a computer system that allows control from two different consoles of a robotic surgical system. Certain complex surgical procedures are better handled by having a first operator and a second operator work in tandem. For these procedures, more than two instruments, in addition to the camera, are attached to the surgical robotic system. The first operator may handle a first set of instruments (and digital applications) and the second operator may handle a second set of instruments (and digital applications).

Each console in the surgical robotic system provides at least two modes of operation. The first mode of operation is a surgical mode in which the operators can control the instruments and the camera through the robotic system. The second mode of operation is an application mode (which may also be referred to as a digital mode) in which the operators may navigate digital menus presented on the console and interact with applications presented on the console. In existing systems, the consoles are restricted to operating in the same mode, which limits functionality and flexibility for the operators. For example, when one console enters the application mode, the other console is also forced into the application mode.

The present disclosure describes a robotic surgical system in which the consoles may be operated in different modes concurrently. For example, one console may operate in the surgical mode while the other console operates in the application mode. The first operator of the first console and the second operator of the second console may seamlessly transition between the surgical mode and the application mode. As a result, the system provides increased functionality and flexibility. For example, the first operator may perform surgical tasks while the second operator handles digital applications. The second operator handles the digital applications without interrupting the surgical tasks of the first operator.

Moreover, when the first console and the second console both use the application mode concurrently, the console that first accessed the application mode is designated as the application mode controller and the other console is designated as the application mode viewer. The application mode controller is allowed to interact with and to change the presentation in the application mode (e.g., launch applications, manipulate application objects, etc.). The application mode viewer may view the same presentation in the application mode as the application mode controller (e.g., see the same application and the results of the manipulations performed by the application mode controller), but the application mode viewer may be restricted from interacting with or changing the presentation in the application mode.

In some embodiments, the system allows the first console and the second console to use different applications in the application mode concurrently. For example, both consoles may interact with and change their presentations in the application mode concurrently if both consoles are accessing different applications. If one of the consoles switches to the same application as the other console, then the system restricts the console to be the application mode viewer and allows the other console to be the application mode controller. In this manner, the system provides even more functionality and flexibility for the operators.

In certain embodiments, the system provides several technical advantages. For example, the system with multiple consoles may facilitate various types of surgeries, especially those involving collaboration among multiple surgeons or medical professionals. Multiple consoles may control the robotic arms concurrently, thus allowing for collaborative surgery. Each console may operate independently which is useful for training purposes or when different aspects of the surgery demand focused attention. The system may further integrate with pre-operative imaging data, thus enabling surgeons to plan and execute procedures with greater accuracy. Therefore, using multiple control consoles advantageously enhances surgical precision, provides for efficient collaboration, better training opportunities, and versatility. Improving surgical precision reduces the risk of errors and complications, and efficient collaboration potentially reduces procedure time. As such, providing multiple consoles offers a versatile and efficient solution for surgical practices, combining the precision of robotics with the expertise of surgeons in a collaborative environment. Thus, the system using multiple consoles may reduce the duration of procedures, improve the health and safety of patients, and reduce recovery times and postoperative complications.

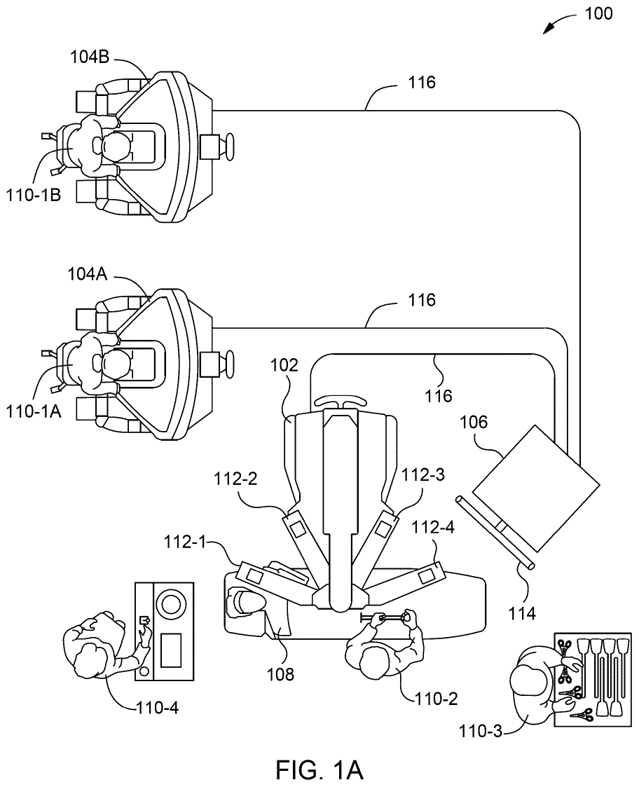

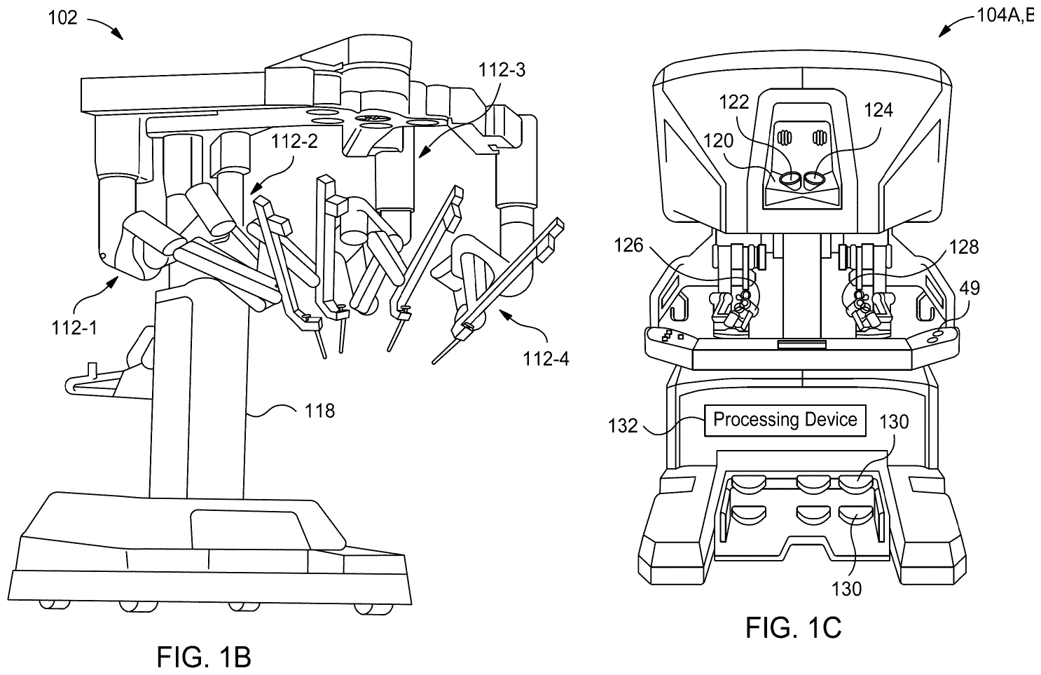

In some examples, one or more components of a medical system may be implemented as a computer-assisted surgical system. It is understood, however, that the medical system may be implemented in any type of medical system (e.g., digital fiducial systems, anatomy detection systems, and clinical guidance systems). FIG. 1A shows an example computer-assisted surgical system 100 that may implement some of the features described herein.

The surgical system 100 includes a manipulator assembly 102, a first user control apparatus 104A, a second user control apparatus 104B, and an auxiliary apparatus 106, all of which are communicatively coupled to each other. The surgical system 100 is utilized by a medical team to perform a computer-assisted medical procedure or other similar operation on a body of a patient 108 or on any other body as may serve a particular implementation. The medical team includes a first control apparatus user 110-1A (such as a surgeon for a surgical procedure), a second control apparatus user 110-1B (such as another surgeon for a surgical procedure), a second user 110-2 (such as a patient-side assistant), a third user 110-3 (such as another assistant, a nurse, a trainee, etc.), and a fourth user 110-4 (such as an anesthesiologist for a surgical procedure), all of whom are collectively referred to as users 110, and each of whom may control, interact with, or otherwise be a user of the surgical system 100. More, fewer, or alternative users may be present during a medical procedure as may serve a particular implementation. For example, team composition for different medical procedures, or for non-medical procedures, may differ and include users with different roles.

Although FIG. 1A illustrates an ongoing minimally invasive medical procedure, it will be understood that the surgical system 100 may similarly be used to perform open medical procedures or other types of operations. For example, operations such as exploratory imaging operations, mock medical procedures used for training purposes, and/or other operations may also be performed.

The manipulator assembly 102 includes one or more manipulator arms 112 (e.g., manipulator arms 112-1 through 112-4) to which one or more instruments may be coupled. The instruments are used for a computer-assisted surgical procedure on the patient 108 (e.g., by being at least partially inserted into the patient 108 and manipulated within the patient 108). While the manipulator assembly 102 is depicted and described herein as including four manipulator arms 112, the manipulator assembly 102 may include a single manipulator arm 112 or any other number of manipulator arms as may serve a particular implementation. Although the example of FIG. 1 illustrates the manipulator arms 112 as robotic manipulator arms, one or more instruments may be partially or entirely manually controlled, such as by being handheld and controlled manually by a person. These partially or entirely manually controlled instruments are used in conjunction with, or as an alternative to, computer-assisted instrumentation that is coupled to the manipulator arms 112.

During the medical operation, the first user control apparatus 104A facilitates tele-operational control by the first control apparatus user 110-1A of the manipulator arms 112 and instruments attached to the manipulator arms 112. To this end, the first user control apparatus 104A provides the first control apparatus user 110-1A with imagery of an operational area associated with the patient 108 as captured by an imaging device. The manipulator arms 112 or any instruments coupled to the manipulator arms 112 mimic the dexterity of the hand, wrist, and fingers of the first control apparatus user 110-1A across multiple degrees of freedom of motion. In this manner, the first control apparatus user 110-1A intuitively performs a procedure (e.g., an incision procedure, a suturing procedure, etc.) using one or more of the manipulator arms 112 or any instruments coupled to the manipulator arms 112.

Similarly, during the medical operation, the second user control apparatus 104B facilitates tele-operational control by the second control apparatus user 110-1B of the manipulator arms 112 and instruments attached to the manipulator arms 112. To this end, the second user control apparatus 104B provides the second control apparatus user 110-1B with imagery of an operational area associated with the patient 108 as captured by an imaging device. The manipulator arms 112 or any instruments coupled to the manipulator arms 112 mimic the dexterity of the hand, wrist, and fingers of the second control apparatus user 110-1B across multiple degrees of freedom of motion. In this manner, the second control apparatus user 110-1B intuitively performs a procedure (e.g., an incision procedure, a suturing procedure, etc.) using one or more of the manipulator arms 112 or any instruments coupled to the manipulator arms 112.

The auxiliary apparatus 106 includes one or more computing devices that perform auxiliary functions in support of the procedure, such as providing insufflation, electrocautery energy, illumination or other energy for imaging devices, image processing, or coordinating components of the surgical system 100. The auxiliary apparatus 106 includes a display monitor 114 that displays one or more user interfaces, or graphical or textual information in support of the procedure. In some instances, the display monitor 114 is a touchscreen display that provides user input functionality. Augmented content provided by a region-based augmentation system may be similar to, or differ from, content associated with the display monitor 114 or one or more display devices in the operation area (not shown).

The manipulator assembly 102, the first user control apparatus 104A, the second user control apparatus 104B, and auxiliary apparatus 106 are communicatively coupled one to another in any suitable manner. The manipulator assembly 102, the first user control apparatus 104A, the second user control apparatus 104B, and auxiliary apparatus 106 may be communicatively coupled by way of control lines 116, which represent any wired or wireless communication link as may serve a particular implementation. To this end, the manipulator assembly 102, the first user control apparatus 104A, the second user control apparatus 104B, and auxiliary apparatus 106 may each include one or more wired or wireless communication interfaces, such as one or more local area network interfaces, Wi-Fi network interfaces, cellular interfaces, and so forth.

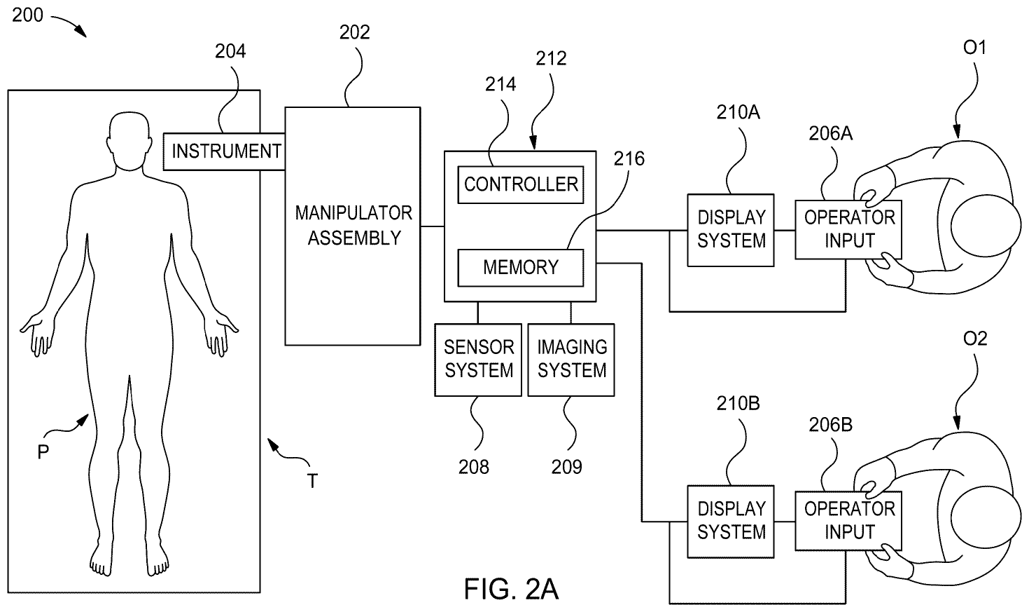

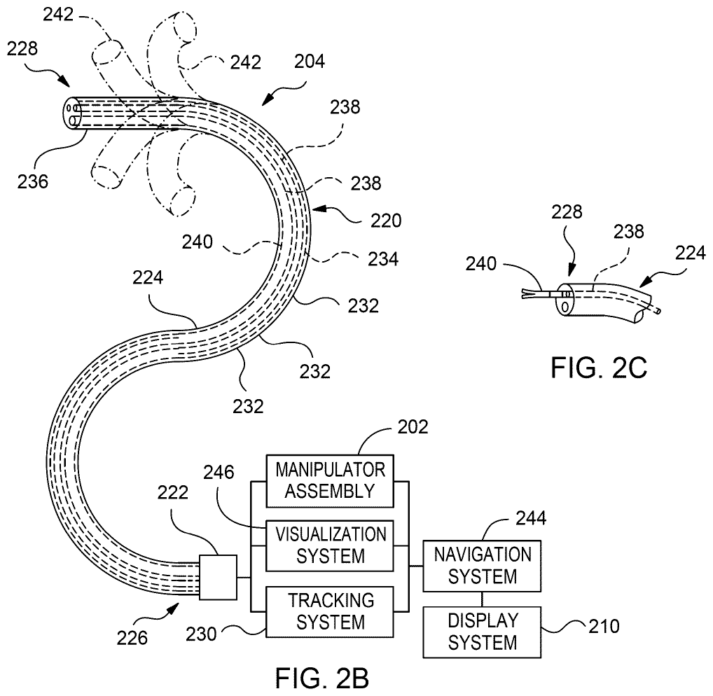

FIG. 1B illustrates an example manipulator assembly 102. As seen in FIG. 2A, the manipulator assembly 102 includes a base 118, a manipulator arm 112-1, a manipulator arm 112-2, a manipulator arm 112-3, and a manipulator arm 112-4. Each manipulator arm 112-1, 112-2, 112-3, and 112-4 is pivotably coupled to the base 118. Although the base 118 may include casters to allow ease of mobility, in some embodiments, the manipulator assembly 102 is fixedly mounted to a floor, ceiling, operating table, structural framework, or the like.

In a typical procedure, two of the manipulator arms 112-1, 112-2, 112-3, or 112-4 hold surgical instruments and a third holds a stereo endoscope. The remaining manipulator arms are available so that other instruments may be introduced at the work site. Alternatively, the remaining manipulator arms may be used for introducing another endoscope or another image capturing device, such as an ultrasound transducer, to the work site.

Each of the manipulator arms 112-1, 112-2, 112-3, and 112-4 are formed of links that are coupled together and manipulated through actuatable joints. Each of the manipulator arms 112-1, 112-2, 112-3, and 112-4 may include a setup arm and a device manipulator. The setup arm positions its held device so that a pivot point occurs at its entry aperture into the patient. The device manipulator may then manipulate its held device so that the held device may be pivoted about the pivot point, inserted into and retracted out of the entry aperture, and rotated about its shaft axis. Each of the manipulator arms 112-1, 112-2, 112-3, and 112-4 may include sensors (e.g., joint sensors, position sensors, accelerometers, etc.) that detect or track movement of the manipulator arms 112-1, 112-2, 112-3, and 112-4. For example, these sensors may detect how far or how quickly a manipulator arm 112-1, 112-2, 112-3, or 112-4 moves in a certain direction.

FIG. 1C illustrates an example user control apparatus 104, which may be used as the first user control apparatus 104A or the second user control apparatus 104B. The user control apparatus 104 includes a stereo vision display 120 so that the user may view the surgical work site in stereo vision from images captured by the stereoscopic camera of the manipulator assembly 102. Left and right eyepieces 122 and 124 are provided in the stereo vision display 120 so that the user may view left and right display screens inside the display 120 respectively with the user's left and right eyes. While viewing typically an image of the surgical site on a suitable viewer or display, the surgeon performs the surgical procedures on the patient by manipulating master control input devices, which in turn control the motion of robotic instruments.

The user control apparatus 104 also includes left and right input devices 126 and 128 that the user grasps respectively with his/her left and right hands to manipulate devices (e.g., surgical instruments) being held by the manipulator arms 112-1, 112-2, 112-3, and 112-3 of the manipulator assembly 102 in preferably six or more degrees of freedom (“DOF”). Foot pedals 130 with toe and heel controls are provided on the user control apparatus 104 so the user may control movement and/or actuation of devices associated with the foot pedals.

A processing device 132 is provided in the user control apparatus 104 for control and other purposes. The processing device 132 performs various functions in the surgical system 100. One function performed by processing device 132 is to translate and transfer the mechanical motion of input devices 126 and 128 to actuate their corresponding joints in their associated manipulator arms 112-1, 112-2, 112-3, and 112-4 so that the surgeon can effectively manipulate devices, such as the surgical instruments. Another function of the processing device 132 is to implement the methods, crosscoupling control logic, and controllers or processors described herein. The auxiliary apparatus 106 may include a processing device 132 that performs the functions or actions described herein. The processing device 132 includes a controller and a memory that perform the functions described herein. The controller may include one or more processors.

The controller may include any electronic circuitry, including, but not limited to one or a combination of microprocessors, microcontrollers, application specific integrated circuits (ASIC), application specific instruction set processor (ASIP), and/or state machines, that communicatively couples to a memory and controls the operation of the user control apparatus 104 and/or the auxiliary apparatus 106. The controller may be 8-bit, 16-bit, 32-bit, 64-bit or of any other suitable architecture. The controller may include an arithmetic logic unit (ALU) for performing arithmetic and logic operations, registers that supply operands to the ALU and store the results of ALU operations, and a control unit that fetches instructions from memory and executes them by directing the coordinated operations of the ALU, registers and other components. The controller may include other hardware that operates software to control and process information. The controller executes software stored on a memory to perform any of the functions described herein. The controller controls the operation and administration of the user control apparatus 104 or the auxiliary apparatus 106 by processing information (e.g., information received from the user control apparatus 104, the manipulator assembly 102, the auxiliary apparatus 106, and/or a memory). The controller is not limited to a single processing device and may encompass multiple processing devices contained in the same device or computer or distributed across multiple devices or computers. The controller is considered to perform a set of functions or actions if the multiple processing devices collectively perform the set of functions or actions, even if different processing devices perform different functions or actions in the set.

FIG. 2A illustrates an example computer-assisted surgical system 200 that implements some of the features described herein. The surgical system 200 can be used, for example, in surgical, diagnostic, therapeutic, biopsy, or non-medical procedures. As shown in FIG. 2A, the surgical system 200 (which may be a robotically-assisted surgical system) includes one or more manipulator assemblies 202 for operating one or more medical instrument systems 204 in performing various procedures on a patient P positioned on a table T in a medical environment. For example, the manipulator assembly 202 can drive catheter or end effector motion, can apply treatment to target tissue, and/or can manipulate control members. The manipulator assembly 202 can be teleoperated, non-teleoperated, or a hybrid teleoperated and non-teleoperated assembly with select degrees of freedom of motion that can be motorized and/or teleoperated and select degrees of freedom of motion that can be non-motorized and/or non-teleoperated. A first operator input system 206A and a second operator input system 206B, which can be inside or outside of the medical environment, generally include one or more control devices for controlling the manipulator assembly 202. The manipulator assembly 202 supports a medical instrument system 204 and can optionally include a plurality of actuators or motors that drive inputs on the medical instrument system 204 in response to commands from a control system 212. The actuators can optionally include drive systems that when coupled to the medical instrument system 204 can advance the medical instrument system 204 into a natural or surgically created anatomic orifice. Other drive systems can move the distal end of the medical instrument in multiple degrees of freedom, which can include three degrees of linear motion (e.g., linear motion along the x, y, and z Cartesian axes) and in three degrees of rotational motion (e.g., rotation about the x, y, and z Cartesian axes). The manipulator assembly 202 can support various other systems for irrigation, treatment, or other purposes. Such systems can include fluid systems (e.g., reservoirs, heating/cooling elements, pumps, and valves), generators, lasers, interrogators, and ablation components.

The surgical system 200 also includes a first display system 210A for displaying an image or representation of the surgical site and a medical instrument system 204. The image or representation is generated by an imaging system 209, which may include an endoscopic imaging system. The first display system 210A and the first operator input system 206A may be oriented so that an operator 01 can control the medical instrument system 204 and the first operator input system 206A with the perception of telepresence. A graphical user interface can be displayable on the first display system 210A and/or a display system of an independent planning workstation.

The surgical system 200 also includes a second display system 210B for displaying an image or representation of the surgical site and a medical instrument system 204. The image or representation is generated by an imaging system 209, which may include an endoscopic imaging system. The second display system 210B and the second operator input system 206B may be oriented so that an operator O2 can control the medical instrument system 204 and the second operator input system 206B with the perception of telepresence. A graphical user interface can be displayable on the second display system 210B and/or a display system of an independent planning workstation.

In some examples, the imaging system 209 includes an endoscopic imaging system with components that are integrally or removably coupled to the medical instrument system 204. However, in some examples, a separate imaging device, such as an endoscope, attached to a separate manipulator assembly can be used with the medical instrument system 204 to image the surgical site. The imaging system 209 can be implemented as hardware, firmware, software, or a combination thereof, which interact with or are otherwise executed by one or more computer processors, which can include the controller 214 of the control system 212.

The surgical system 200 also includes a sensor system 208. The sensor system 208 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 204. These sensors may also detect a position, orientation, or pose of the patient P on the table T. For example, the sensors may detect whether the patient P is face-down or face-up. As another example, the sensors may detect a direction in which the head of the patient P is directed. The sensor system 208 can also include temperature, pressure, force, or contact sensors, or the like.

The surgical system 200 can also include a control system 212, which includes at least one memory 216 and at least one controller 214 (which may include a processor) for effecting control between the medical instrument system 204, the first operator input system 206A, the second operator input system 206B, the sensor system 208, the first display system 210A, and the second display system 210B. The control system 212 includes programmed instructions (e.g., a non-transitory machine-readable medium storing the instructions) to implement a procedure using the surgical system 200, including for navigation, steering, imaging, engagement feature deployment or retraction, applying treatment to target tissue (e.g., via the application of energy), or the like.

The control system 212 may further include a virtual visualization system to provide navigation assistance to the operator O when controlling medical instrument system 204 during an image-guided surgical procedure. Virtual navigation using the virtual visualization system can be based upon reference to an acquired pre-operative or intra-operative dataset of anatomic passageways. The virtual visualization system processes images of the surgical site imaged using imaging technology, such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 212 uses a pre-operative image to locate the target tissue (using vision imaging techniques and/or by receiving user input) and create a pre-operative plan, including an optimal first location for performing treatment. The pre-operative plan can include, for example, a planned size to expand an expandable device, a treatment duration, a treatment temperature, and/or multiple deployment locations.

The controller 214 is any electronic circuitry, including, but not limited to one or a combination of microprocessors, microcontrollers, application specific integrated circuits (ASIC), application specific instruction set processor (ASIP), and/or state machines, that communicatively couples to the memory 216 and controls the operation of the control system 212. The controller 214 may be 8-bit, 16-bit, 32-bit, 64-bit or of any other suitable architecture. The controller 214 may include an arithmetic logic unit (ALU) for performing arithmetic and logic operations, processor registers that supply operands to the ALU and store the results of ALU operations, and a control unit that fetches instructions from memory and executes them by directing the coordinated operations of the ALU, registers and other components. The controller 214 may include other hardware that operates software to control and process information. The controller 214 executes software stored on the memory 216 to perform any of the functions described herein. The controller 214 controls the operation and administration of the control system 212 by processing information (e.g., information received from the manipulator assembly 202, the first operator input system 206A, the second operator input system 206B, and the memory 216). The controller 214 is not limited to a single processing device and may encompass multiple processing devices contained in the same device or computer or distributed across multiple devices or computers. The controller 214 is considered to perform a set of functions or actions if the multiple processing devices collectively perform the set of functions or actions, even if different processing devices perform different functions or actions in the set.

The memory 216 may store, either permanently or temporarily, data, operational software, or other information for the controller 214. The memory 216 may include any one or a combination of volatile or non-volatile local or remote devices suitable for storing information. For example, the memory 216 may include random access memory (RAM), read only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory 216, a disk, a CD, or a flash drive. In particular embodiments, the software may include an application executable by the controller 214 to perform one or more of the functions described herein. The memory 216 is not limited to a single memory and may encompass multiple memories contained in the same device or computer or distributed across multiple devices or computers. The memory 216 is considered to store a set of data, operational software, or information if the multiple memories collectively store the set of data, operational software, or information, even if different memories store different portions of the data, operational software, or information in the set.

FIG. 2B illustrates an example medical instrument system 204 in the surgical system 200. In some embodiments, the medical instrument system 204 is used in an image-guided medical procedure. For example, the medical instrument system 204 may be used for non-teleoperational exploratory procedures or in procedures involving traditional manually operated medical instruments, such as endoscopy.

The medical instrument system 204 includes an elongate flexible device 220, such as a flexible catheter or endoscope (e.g., gastroscope, bronchoscope), coupled to a drive unit 222. The elongate flexible device 220 includes a flexible body 224 having a proximal end 226 and a distal end, or tip portion, 228. In some embodiments, the flexible body 224 has an approximately 14-20 millimeter outer diameter. Other flexible body outer diameters may be larger or smaller. The flexible body 224 has an appropriate length to reach certain portions of the anatomy, such as the lungs, sinuses, throat, or the upper or lower gastrointestional region, when the flexible body 224 is inserted into a patient's oral or nasal cavity.

The medical instrument system 204 includes a tracking system 230 for determining the position, orientation, speed, velocity, pose, and/or shape of the distal end 228 and/or of one or more segments 232 along the flexible body 224 using one or more sensors and/or imaging devices. The entire length of the flexible body 224, between the distal end 228 and the proximal end 226, is effectively divided into the segments 232. The tracking system 230 is implemented as hardware, firmware, software, or a combination thereof, which interact with or are otherwise executed by one or more computer processors, which may include the controller 214 of control system 212.

The tracking system 230 tracks distal end 228 and/or one or more of the segments 232 using a shape sensor 234. In some embodiments, the tracking system 230 tracks the distal end 228 using a position sensor system 236, such as an electromagnetic (EM) sensor system. In some examples, the position sensor system 236 measures six degrees of freedom (e.g., three position coordinates x, y, and z and three orientation angles indicating pitch, yaw, and roll of a base point) or five degrees of freedom (e.g., three position coordinates x, y, and z and two orientation angles indicating pitch and yaw of a base point).

The flexible body 224 includes one or more channels 238 sized and shaped to receive one or more medical instruments 240. In some embodiments, the flexible body 224 includes two channels 238 for separate medical instruments 240, however, a different number of channels 238 can be provided. FIG. 2C illustrates an example portion of the medical instrument system 204 of FIG. 2B. As seen in FIG. 2C, the medical instrument 240 extends through the flexible body 224. In some embodiments, the medical instrument 240 can be used for procedures and aspects of procedures, such as surgery, biopsy, ablation, mapping, imaging, illumination, irrigation, or suction. The medical instrument 240 is deployed through the channel 238 of the flexible body 224 and is used at a target location within the anatomy. The medical instrument 240 includes, for example, image capture devices, biopsy instruments, ablation instruments, catheters, laser ablation fibers, and/or other surgical, diagnostic, or therapeutic tools. The medical tools include end effectors having a single working member such as a scalpel, a blunt blade, a lens, an optical fiber, an electrode, and/or the like. Other end effectors include, for example, forceps, graspers, balloons, needles, scissors, clip appliers, and/or the like. Other end effectors further include electrically activated end effectors such as electrosurgical electrodes, transducers, sensors, imaging devices, and/or the like. The medical instrument 240 is advanced from the opening of the channel 238 to perform the procedure and then retracted back into the channel when the procedure is complete. The medical instrument 240 is removed from the proximal end 226 of the flexible body 224 or from another optional instrument port (not shown) along the flexible body 224. The medical instrument 240 may be used with an image capture device (e.g., an endoscopic camera) also within the elongate flexible device 220. Alternatively, the medical instrument 240 may itself be the image capture device.

The medical instrument 240 additionally houses cables, linkages, or other actuation controls (not shown) that extend between the proximal and distal ends to controllably bend the distal end of the medical instrument 240. The flexible body 224 also houses cables, linkages, or other steering controls (not shown) that extend between the drive unit 222 and the distal end 228 to controllably bend the distal end 228 as shown, for example, by the broken dashed line depictions 242 of the distal end 228. In some examples, at least four cables are used to provide independent “up-down” steering to control a pitch motion of the distal end 228 and “left-right” steering to control a yaw motion of the distal end 228. In embodiments in which the medical instrument system 204 is actuated by a robotically-assisted assembly, the drive unit 222 can include drive inputs that removably couple to and receive power from drive elements, such as actuators, of the teleoperational assembly. In some embodiments, the medical instrument system 204 includes gripping features, manual actuators, or other components for manually controlling the motion of the medical instrument system 204. The information from the tracking system 230 can be sent to a navigation system 244, where the information is combined with information from the visualization system 246 and/or the preoperatively obtained models to provide the physician or other operator with real-time position information.

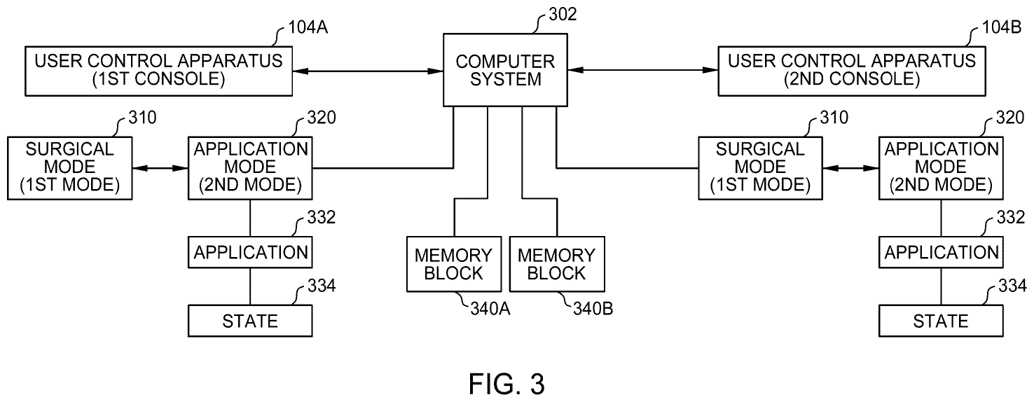

FIGS. 3 through 12B illustrate example operations performed by a medical system (e.g., the surgical system 100 of FIG. 1A or the surgical system 200 of FIG. 2A). Generally, using these operations, a computer system (which may be implemented using the auxiliary apparatus 106 of the surgical system 100 (e.g., using the processing device 132 of the auxiliary apparatus 106) or in the control system 212 of the surgical system 200 using the controller 214 and the memory 216) in the medical system allows control from two different consoles 104 of the medical system.

FIG. 3 illustrates example mode transitions using the computer system 302 (which may be implemented using the auxiliary apparatus 106 of the surgical system 100 or in the control system 212 of the surgical system 200), the first user control apparatus 104A, and the second user control apparatus 104B. The first user control apparatus 104A may also be referred to as a first console 104A and the second user control apparatus 104B may also be referred to as a second console 104B.

Each of the first console 104A and the second console 104B has two modes of operation that are tracked by the computer system 302. The first mode of operation is a surgical mode 310 in which the operators may control the surgical instruments and the camera through the robotic system. The second mode of operation is an application mode 320 (which may also be referred to as a digital mode) in which the operators may navigate digital menus presented on the consoles and interact with applications presented on the consoles. The first console 104A and the second console 104B may transition between the surgical mode 310 and the application mode 320 based on input provided by the users at the first console 104A and the second console 104B (e.g., using input devices 126 and/or 128, foot pedals 130, and/or operator input 206A and 206B). The computer system 302 receives the input from the first console 104A and the second console 104B and tracks the modes of operation of the first console 104A and the second console 104B. The computer system 302 communicates videos to the consoles 104A and 104B according to whether the consoles 104A and 104B are in the surgical mode 310 or the application mode 320. For example, in the surgical mode 310, the computer system 302 communicates videos of the surgical site to the consoles 104A and 104B. In the application mode 320, the computer system 302 communicates videos of applications.

In the application mode 320, the consoles 104A and 104B may be used to launch and interact with one or more applications 332. Interacting with an application 332 may change a state 334 of the application 332. The application 332 of the first console 104A (along with its corresponding state 334) may be stored in a first memory block 340A and the application 332 of the second console 104B (along with its corresponding state 334) may be stored in a second memory block 340B. In the example of FIG. 3, the computer system 302 stores the application 332 and state 334 for each console 104 in a separate memory block 340. However, in another example, the computer system 302 may store the application 332 and state 334 for both consoles 104 in a shared memory block (not shown). As the computer system 302 receives inputs from the consoles 104A and 104B to launch and interact with applications 332, the computer system 302 communicates updated videos to the consoles 104A and 104B to show the launching and interactions with the applications 332.

The applications 332 may provide different features. For example, an application 332 may track instruments in the surgical space and visualize positions of instruments in the surgical space (e.g., on a model or a map). The consoles 104A and 104B may interact with this application by moving or rotating the model, which changes the state 334 of the application 332. As another example, an application 332 may provide visuals of the imaging space and determine optimal incision locations. The consoles 104A and 104B may interact with this application by placing markings on an anatomical structure in the surgical space to indicate where incisions were or should be made. The application 332 may thus provide capabilities or include features related to at least three-dimensional (3D) visualization, image fusion, image registration, target localization, real-time tracking, intraoperative imaging, and image annotation and marking.

In existing systems, consoles are restricted to operating in the same mode, which limits functionality and flexibility for the operators. For example, when one console enters the application mode 320, the other console is also forced into the application mode 320. In contrast, the surgical system 100 and the surgical system 200 allow the first console 104A and the second console 104B to operate in different modes. For example, the first console 104A may operate in the surgical mode 310 while the second console 104B operates in the application mode 320. The first console 104A and the second console 104B may seamlessly transition between the surgical mode 310 and the application mode 320. As a result, the surgical system 100 and the surgical system 200 provide increased functionality and flexibility. For example, the first operator may perform surgical tasks while the second operator may handle digital applications. The second operator may handle the digital applications without interrupting the surgical tasks of the first operator.

This disclosure contemplates a variety of options and operations when both consoles 104A and 104B operate in the application mode 320 at the same time. In one example, the consoles 104A and 104B have separate and independent application spaces. Both consoles 104A and 104B may launch their own instances of applications 332 in the independent application spaces. The states 334 of these applications 332 are also kept independent. For example, even if both consoles 104A and 104B launch instances of the same application 332, the consoles 104A and 104B may allow the states 334 of these instances to be changed independently of one another.

In another example, the consoles 104A and 104B may share the same application space. Whichever console first enters the application mode 320 takes control of the application space, and the other console may be designated as a spectator that is not allowed to control the application space. For example, if the console 104A first enters the application mode 320, then the console 104A may be given control to launch and interact with applications 332. If the console 104B subsequently enters the application mode 320 while the console 104A is still in the application mode 320, then the console 104B may view what the console 104A views (e.g., view the same application 332 and the same state 334). If the console 104A leaves the application mode 320 while the console 104B remains in the application mode 320, then the console 104B may be given control of the application space.

As another example, the consoles 104A and 104B may share instances for the same application. Whichever console first enters an application 332 takes control of the application 332, and the other console may be designated as a spectator that is not allowed to control the application 332. For example, if the console 104A first enters an application 332, then the console 104A may be given control to launch and interact with that application 332. If the console 104B subsequently enters that same application 332 while the console 104A is still in control of that application 332, then the console 104B may view what the console 104A views (e.g., view the same application 332 and the same state 334). If the console 104A leaves the application 332 (e.g., launches another application or leaves the application mode 320) while the console 104B remains in the application 332, then the console 104B may be given control of the application 332. The console 104B may navigate to a different application and be given control of that application while the console 104A retains control of the original application.

As an example operation, the first console 104A may be in the surgical mode 310, and the second console 104B may also be in the surgical mode 310. If the first operator of the first console 104A transitions to the application mode 320, the first operator may select an application 332. The application 332 may provide a pre-operative image. The pre-operative image may assist the first operator in placing markings on an anatomical structure in the surgical space. The state 334 of the application 332 includes the placed markers. The first operator of the first console 104A may transition back to the surgical mode 310 after the markers have been placed. If the first operator subsequently transitions back to the application mode 320, the first console 104A may show the application 332 with the latest state 334 (e.g., showing the previously placed markers). In some embodiments, the first console 104A first offers the first operator the option to view the application 332 in the latest state 334 or to access another application. If the first operator chooses to view the application 332 in the latest state 334, the first console 104A may then show the pre-operative image with the previously placed markers. If the first operator choose to access another application, then the first console 104A may allow the first operator to select another application to launch. Therefore, the first operator of the first console 104A may seamlessly transition between different modes. Also, the first operator of the first console 104A may operate in one mode while the second operator of the second console 104B operates in a different mode.

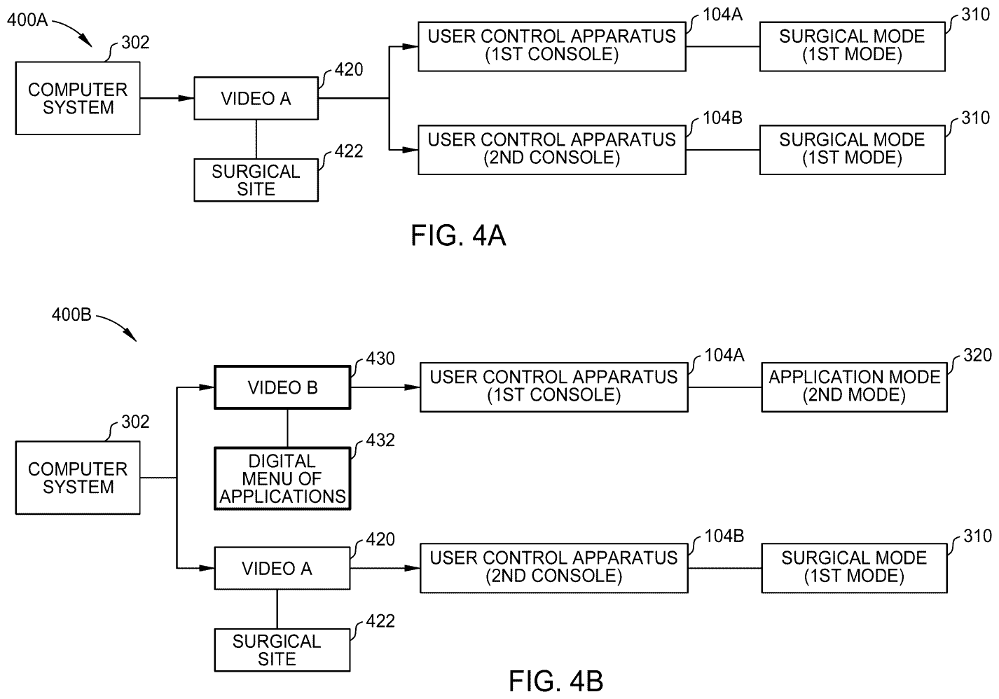

FIGS. 4A and 4B illustrate example operations 400A and 400B performed by the medical system. Generally, a computer system 302 and consoles 104 perform the operations 400A and 400B to allow control from two different consoles.

In operation 400A, the computer system 302 communicates, to the first console 104A and the second console 104B, a first video 420 (video A) captured by a camera. The first video 420 is a video of a surgical site 422. The first console 104A and the second console 104B may both be in the surgical mode 310. The first video 420 of the surgical site 422 is communicated to the first console 104A and the second console 104B based on the computer system 302 determining that both the first console 104A and the second console 104B are operating in the surgical mode 310.

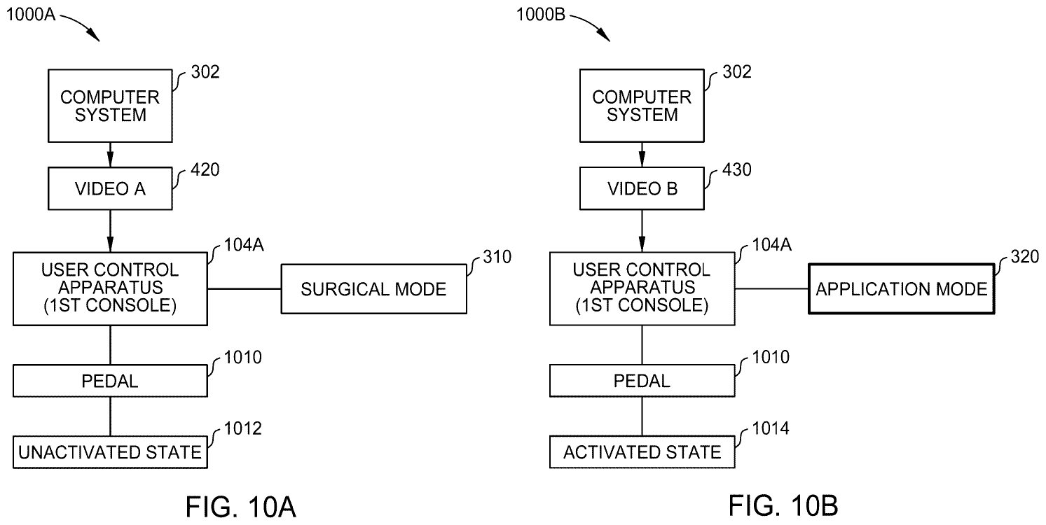

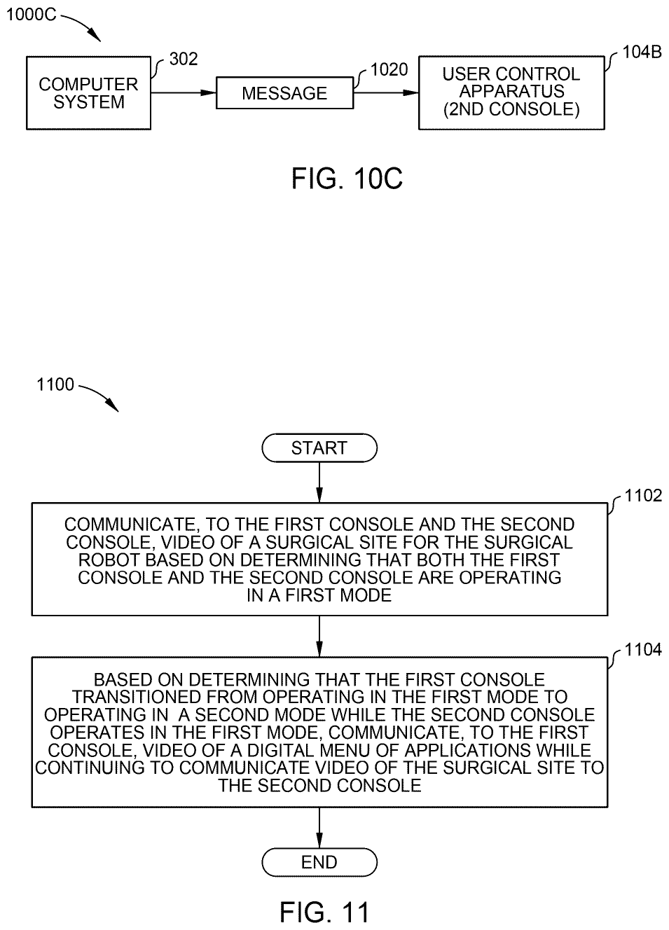

In operation 400B, the first console 104A transitions from the surgical mode 310 to the application mode 320. The transition from the surgical mode 310 to the application mode 320 may be triggered by pressing a pedal on the first console 104A, as described below with reference to FIGS. 10A and 10B. Based on determining that the first console 104A transitioned from operating in the surgical mode 310 to operating in the application mode 320 while the second console 104B operates in the surgical mode 310, the computer system 302 communicates, to the first console 104A, a second video 430 (video B) of a digital menu of applications 432 while continuing to communicate the first video 420 of the surgical site 422 to the second console 104B.

Therefore, when the first console 104A transitions to the application mode 320, the second console 104B remains in the surgical mode 310. The mode transition of the first console 104A does not force the second console 104B into the application mode 320. The first operator of the first console 104A may select an application 332 from the digital menu of applications 432 in the application mode 320, while the operator of the second console 104B remains in the surgical mode 310. The surgical tasks performed by the second operator of the second console 104B remain uninterrupted while the first console 104A is in the application mode 320. Therefore, the first console 104A and the second console 104B may operate in different modes.

Stated differently, one console may operate in the surgical mode 310 while the other console operates in the application mode 320. The first operator of the first console 104A and the second operator of the second console 104B may seamlessly transition between the surgical mode 310 and the application mode 320. As a result, the computer system 302 provides increased functionality and flexibility. For example, the first operator may perform surgical tasks while the second operator handles digital applications. The second operator may handle the digital applications without interrupting the surgical tasks of the first operator.

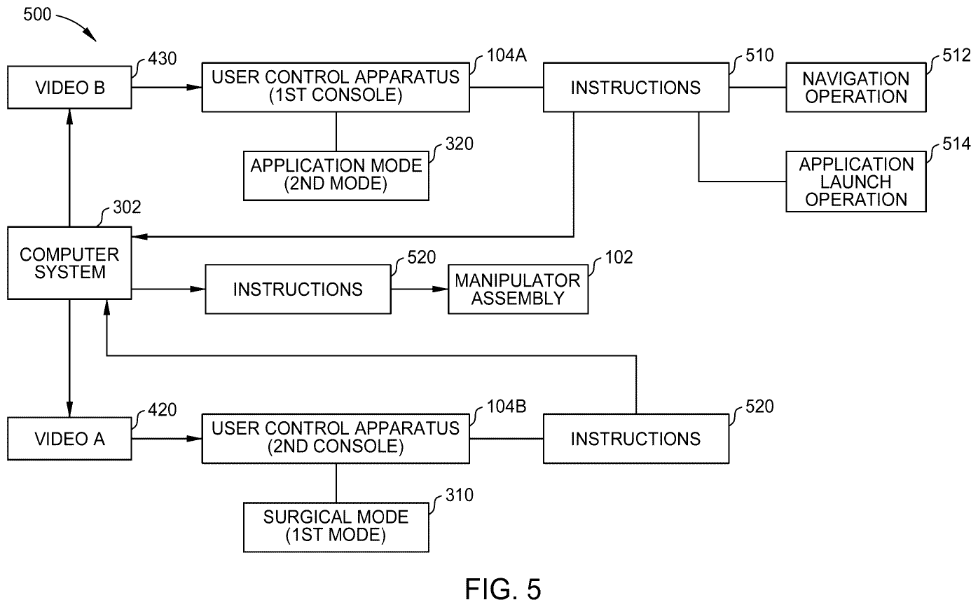

FIG. 5 illustrates an example operation 500 performed by the medical system. Generally, the computer system 302 and the consoles 104 perform the operation 500 to allow the first console 104A to transition to the application mode 320 to navigate a digital menu of applications 432 and to launch an application therefrom.

When the first console 104A transitions to the application mode 320, the first console 104A receives, from the computer system 302, the second video 430 to access the digital menu of applications 432. The first console 104A may send instructions 510 to the computer system 302 based on input from a first operator of the first console 104A. The instructions 510 may instruct the computer system 302 to perform a navigation operation 512, which navigates the digital menu. The instructions 510 may also instruct the computer system 302 to perform an application launch operation 514 to launch an application from the digital menu. Once the application is launched, the computer system 302 updates the second video 430 to show the launched application. The computer system communicates the updated second video 430 to the first operator of the first console 104A. The launched application may then be displayed on the first console 104A.

The computer system 302 continues to communicate the first video 420 to the second console 104B, which remains in the surgical mode 310. The second console 104B communicates instructions 520 to the computer system 412 to operate the surgical robot. The instructions 520 may be generated according to input provided by the second operator of the second console 104B. The computer system 302 communicates the instructions 520 to the surgical robot (e.g., to a manipulator assembly 102) to control the surgical robot according to the input provided by the second operator. Therefore, as the first console 104A is navigating the digital menu of applications 432 to select an application, the second console 104B may continue in the surgical mode 310 to perform surgical tasks at the surgical site 422. For example, the second console 104B may continue controlling the manipulator assembly 102 of the surgical system 100. As a result, the first console 104A and the second console 104B may operate in different modes. The surgical tasks performed by the second operator of the second console 104B remain uninterrupted while the first console 104A is in the application mode 320. The first console 104A and the second console 104B may thus transition between the surgical mode 310 and the application mode 320 independently of each other. As a result, the surgical system 100 may provide increased functionality and flexibility.

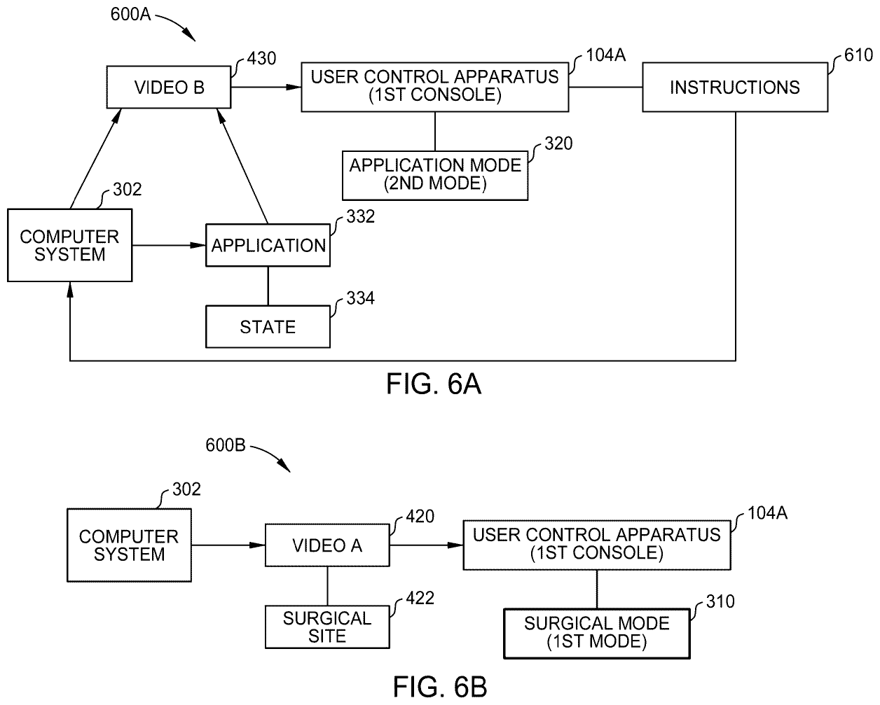

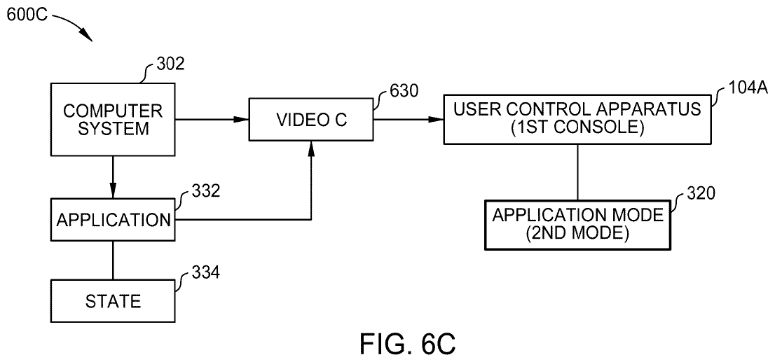

FIGS. 6A through 6C illustrate example operations performed by the medical system. Generally, the computer system 302 and the consoles 104 perform the operations 600A through 600C to allow the first console 104A to transition from the application mode 320 to the surgical mode 310 and to change the state of the launched application.

In operation 600A shown in FIG. 6A, when the first console 104A transitions to the application mode 320, the computer system 302 communicates, to the first console 104A, the second video 430 to access the digital menu of applications 432. The first console 104A may send instructions 610 to the computer system 302 to launch an application 332. After the computer system 302 launches the application 332, the computer system 302 updates the second video 430 to show the launched application 332. The instructions 610 may also include interactions and manipulations to the application 332 (e.g., based on user input from the first operator of the first console 104A) that change the state 334 of the application 332. As an example, if the application 332 shows a pre-operative image of the surgical site, then the manipulations and interactions may include markers placed on the pre-operative image or adjustments (e.g., rotations, translations, etc.) to the pre-operative image to show a different perspective of the surgical site.

In one example, the first operator of the first console 104A transitions from the surgical mode 310 to the application mode 320. In the application mode 320, the first operator navigates the digital menu of applications 432, which shows multiple applications. The first operator selects one of the applications, which is an application that has not been previously launched by the first console 104A during the medical procedure. As an example, the selected application may provide a pre-operative image of the surgical site to assist the first operator. The first console 104A communicates instructions 610 to the computer system 302 to launch the selected application, and the computer system 302 launches the application. The computer system 302 then updates the second video 430 to display the launched application (e.g., to show the pre-operative image of the surgical site).

If the first operator of the first console 104A transitions back from the surgical mode 310 to the application mode 320 at a subsequent time and accesses the same application, such application would retain the state 334. As a result, the computer system 302 may show the application in the same state as when the first operator transitioned away from the application mode 320, rather than show the application in an initial, launched state.

In operation 600B shown in FIG. 6B, after launching the application and determining that the first console 104A transitioned from operating in the application mode 320 back to the surgical mode 310, the computer system 302 communicates the first video 420 of the surgical site 422 to the first console 104A.

After the first operator of the first console 104A finishes using the application, the first operator transitions the first console 104A from the application mode 320 back to the surgical mode 310 to perform further surgical tasks. When the first console 104A transitions back to the surgical mode 310, the computer system 302 communicates the first video 420 of the surgical site 422 to the first console 104A. The first video 420 may have been updated to show any changes to the surgical site 422 that occurred while the first console 104A was in the application mode 320.

In operation 600C shown in FIG. 6C, the first console 104A transitions back to the application mode 320 after transitioning to the surgical mode 310. Because the first console 104A previously launched the application 332 in the application mode 320, the computer system 302 communicates a third video 630 (video C) to the first console 104A. The third video 630 is video of the launched application 332. As a result, the first console 104A may show the launched application 332 upon transitioning back to the application mode 320 (as opposed to the digital menu of applications 432). Additionally, the state 334 of the application 332 may be preserved. For example, any interactions or manipulations that the first operator made to the launched application 332 (e.g., previously placed markers, adjustments to a pre-operative image, etc.) may be preserved and shown in the third video 630.

Stated differently, when the first console 104A transitions from the surgical mode 310 back to the application mode 320 at a subsequent time, the third video 630 of the launched application is provided to the first console 104A. As such, once the first console 104A re-enters the application mode 320 at a subsequent time, the previously launched application 332 is automatically provided to the first console 104A. Using the previous example of the application that shows the pre-operative image, the third video 630 will show the application and the pre-operative image on the first console 104A when the first console 104A re-enters the application mode 320. Any manipulations or interactions that the first console 104A made may also be shown in the third video 630. As such, when the first console 104A returns to the application mode 320 from the surgical mode 310, the last state 334 of the application mode 320 is presented to the first operator of the first console 104A, rather than an initial home screen.

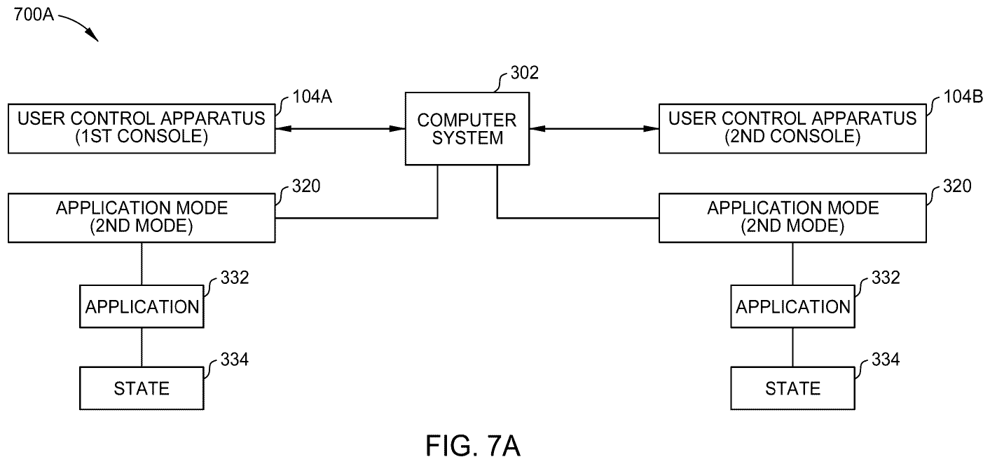

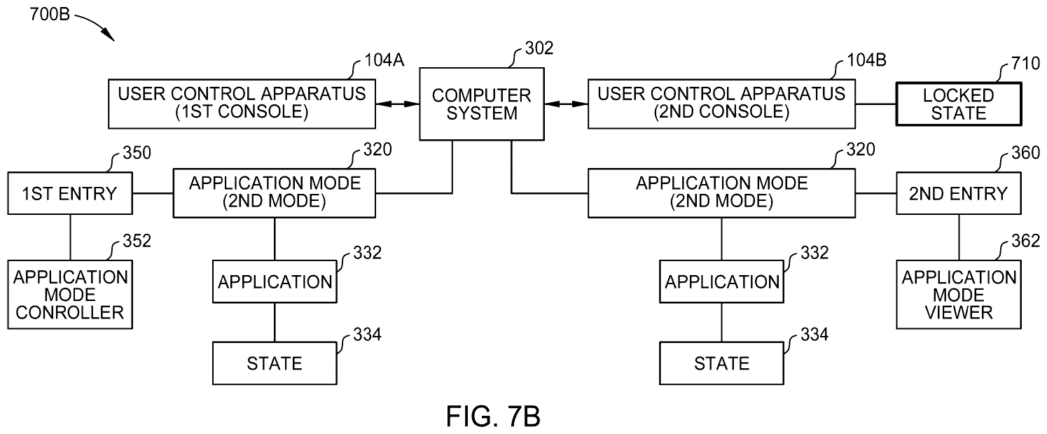

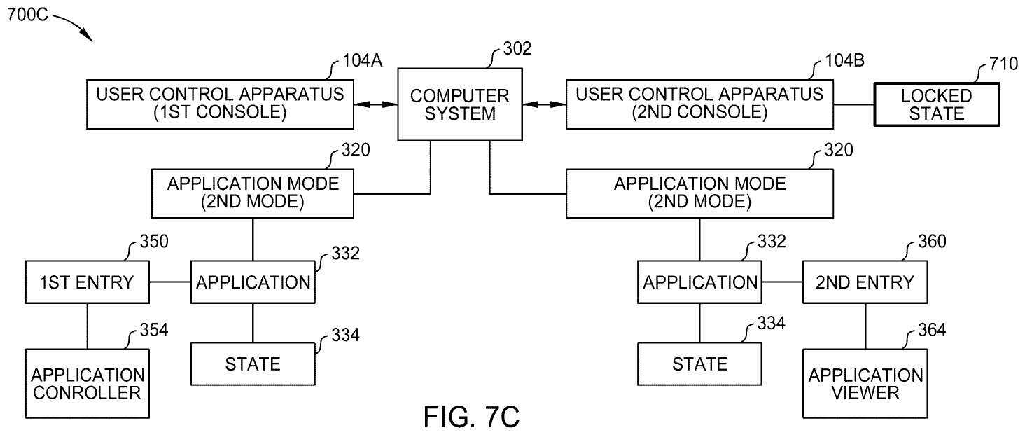

FIGS. 7A through 7C illustrate example operations 700A, 700B, and 700C performed by the medical system. The computer system 302 and the consoles 104 perform the operations 700A, 700B, and 700C to implement different functionality when the consoles 104 are both in the application mode 320. For example, the computer system 302 may implement separate and independent application spaces for the consoles 104A and 104B. As another example, the computer system 302 may implement shared application spaces for the consoles 104A and 104B. As another example, the computer system 302 may implement shared application instances for the consoles 104A and 104B. Generally, each operation 700A, 700B, and 700C begins with both consoles 104A and 104B in the application mode 320. For example, the first console 104A may transition to the application mode 320 first, followed by the second console 104B.

In the operation 700A, the computer system 302 implements separate and independent application spaces for the consoles 104A and 104B. As a result, the actions of the first console 104A in the application mode 320 generally do not affect what the computer system 302 communicates to the second console 104B in the application mode 320. Both consoles 104A and 104B may launch their own instances of applications 332. The states 334 of these applications 332 are also kept independent. For example, the consoles 104A and 104B may launch different applications 332, and the computer system 302 may communicate different videos showing these applications 332 to the consoles 104A and 104B. As another example, even if both consoles 104A and 104B launch the same application 332, the computer system 302 communicates different videos showing different instances of the application 332 to the consoles 104A and 104B. The computer system 302 allows the states 334 of these instances to be changed and maintained independently of one another.

As an example operation, the first console 104A may interact with an application 332 and the second console may interact with a different application 332. Interacting with an application 332 may change a state 334 of the application 332. However, the state 334 of the application 332 used by the first console 104A changes independently of the state 334 of the application 332 used by the second console 104B. Stated differently, the states 334 of the applications 332 are independent from one another. Similarly, the first console 104A may interact with an application 332 and the second console may interact with the same application 332. Again, the state 334 of the application 332 used by the first console 104A changes independently of the state 334 of the application 332 used by the second console 104B even though both consoles 104 use the same application 332. Thus, the state 334 of the application 332 for each console 104 also changes independently.

In the operation 700B shown in FIG. 7B, the computer system 302 implements a shared application space for the consoles 104A and 104B. The console 104A that first enters the application mode 320 takes control of the application space, and the other console 104B may be designated as a spectator that is not allowed to control the application space (e.g., until the console 104A transitions back to the surgical mode 310). For example, the console 104A may be given control to launch and interact with applications 332. The console 104B may view what the console 104A views (e.g., view the same application 332 and the same state 334). If the console 104A leaves the application mode 320 while the console 104B remains in the application mode 320, then the console 104B may be given control of the application space.

As an example operation, the first console 104A makes a first entry 350 into the application mode 320. The second console 104B makes a second entry 360 into the application mode 320. When the second console 104B makes the second entry 360 into the application mode 320, the first console 104A becomes the application mode controller 352 and the second console 104B becomes the application mode viewer 362. The first console 104A, as the application mode controller 352, is permitted to interact with and change the presentation in the application mode 320. The second console 104B, as the application mode viewer 362, is restricted from interacting with or changing the presentation in the application mode 320. The second console 104B is thus in a locked state 710. As such, in the operation 700B, the first console 104 to access the application mode 320 becomes the application mode controller 352 and controls what is viewed by the other console 104 subsequently accessing the application mode 320. The application mode controller 352 is allowed to interact with and to change the presentation in the application mode 320 (e.g., launch applications, manipulate application objects, etc.). The application mode viewer 362 may view the same presentation in the application mode 320 as the application mode controller (e.g., see the same application and the results of the manipulations performed by the application mode controller), but the application mode viewer 362 is restricted from interacting with or changing the presentation in the application mode 320. As a result, in the operation 700B, the computer system 302 communicates the same video to both consoles 104A and 104B when both consoles 104A and 104B are in the application mode 320.

In the operation 700C shown in FIG. 7C, the computer system 302 implements shared application instances for the consoles 104A and 104B. The console 104A that first accesses an application 332 takes control of the application 332, and if the other console 104B accesses the same application 332 when the console 104A is accessing the application 332, then the other consoles 104B is designated as a spectator that is not allowed to control the application 332. For example, if the console 104A first accesses an application 332, then the console 104A may be given control to interact with that application 332. If the console 104B subsequently accesses that same application 332 while the console 104A is still in control of that application 332, then the console 104B may view what the console 104A views (e.g., view the same application 332 and the same state 334). If the console 104A leaves the application 332 (e.g., launches another application or leaves the application mode 320) while the console 104B remains in the application 332, then the console 104B may be given control of the application 332. If the consoles 104A and 104B access different applications 332, then the consoles 104A and 104B may control and interact with their respective applications 332. It is when the consoles 104A and 104B access the same application 332 concurrently that the computer system 302 gives one console 104A control while locking out the other console 104B.

As an example operation, the first console 104A makes a first entry 350 into the application 332. The second console 104B makes a second entry 360 into the same application 332. When the second console 104B makes the second entry 360 into the application 332, the first console 104A becomes the application controller 354 and the second console 104B becomes the application viewer 364. The first console 104A, as the application controller 354, is permitted to interact with and change the state 334 of the application 332. The second console 104B, as the application viewer 364, is restricted from interacting with or changing the state 334 of the application 332. The second console 104B is thus in a locked state 710. As such, in the operation 700C, the first console 104A to access the application 332 becomes the application controller 354 and controls what is viewed by the other console 104B subsequently accessing the application 332. The application controller 354 is allowed to interact with and to change the state 334 of the application 332. The application viewer 364 may view the same application 332, but the application viewer 364 is restricted from interacting with or changing the state 334.

The computer system 302 thus prevents the second console 104B from changing the state 334 of the application 332 while the first console 104A and the second console 104B are both accessing the same application 332. If the consoles 104A and 104B access different applications 332, then both consoles 104A and 104B are given control and interact with their respective applications 332. As a result, the computer system 302 communicates different videos showing different applications when the consoles 104A and 104B access different applications. When the consoles 104A and 104B access the same application 332, then the computer system 302 communicates the same video to both consoles 104A and 104B.

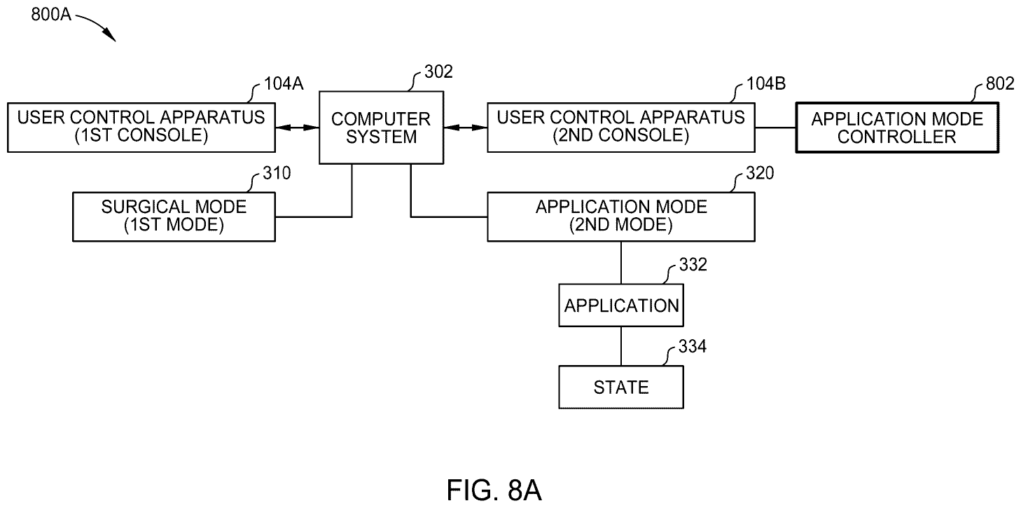

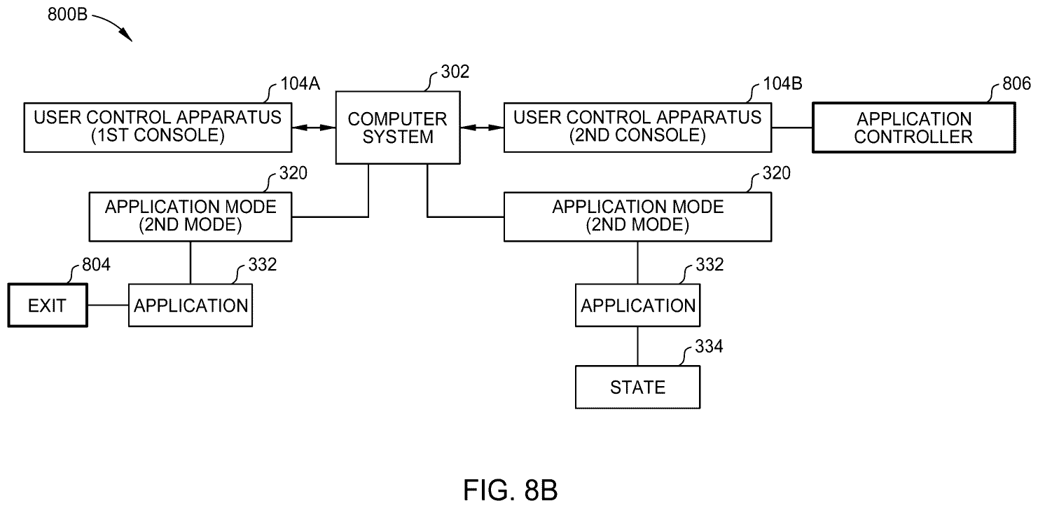

FIG. 8A illustrates an example operation 800A performed by the medical system. Generally, the computer system 302 and the consoles 104A and 104B perform the operation 800A when the computer system 302 implements a shared application space for the consoles 104A and 104B (as described using operation 700B in FIG. 7B). In the shared application space, the console 104A that first transitions to the application mode 320 is given control, and the other console 104B that subsequently transitions to the application mode 320 acts as a viewer. By performing the operation 800A, the computer system 302 transitions the second console 104B to become the application mode controller 802 when the first console 104A exits the application mode 320.

The operation 800A shown in FIG. 8A begins with the first console 104A and the second console 104B operating in the application mode 320, and then the first console 104A transitions back to the surgical mode 310. When the first console 104A enters the surgical mode 310, the computer system 302 allows the second console 104B to become the application mode controller 802.

The second console 104B, as the application mode controller 802, is permitted to interact with and change the presentation in the application mode 320. If the first console 104A subsequently enters the application mode 320, the first console 104A becomes the application mode viewer. The first console 104A, as the application mode viewer, is restricted from interacting with or changing the presentation in the application mode 320. The first console 104A is thus in a locked state. The application mode viewer may view the same presentation in the application mode 320 as the application mode controller 802 (e.g., see the same application and the results of the manipulations performed by the application mode controller 802).

FIG. 8B illustrates an example operation 800B performed by the medical system. Generally, the computer system 302 and the consoles 104A and 104B perform the operation 800B when the computer system 302 implements shared application instances for the consoles 104A and 104B (as described using operation 700C in FIG. 7C). With shared application instances, the console 104A that first accesses an application 332 is given control, and the console 104B that subsequently accesses that application 332 acts as a viewer. By performing the operation 800B, the computer system 302 transitions the second console 104B to become the application controller 806 when the first console 104A exits 804 the application 332 (e.g., the console 104A may access a different application 332, or the console 104A may transition from the application mode 320 to the surgical mode 310).

The second console 104B, as the application controller 806, is permitted to interact with and change the state 334 of the application 332. If the first console 104A subsequently accesses the application 332 when the second console 104B is the application controller 806, the first console 104A becomes the application viewer. The first console 104A, as the application viewer, is restricted from interacting with or changing the state 334 of the application 332. The first console 104A is thus in a locked state.

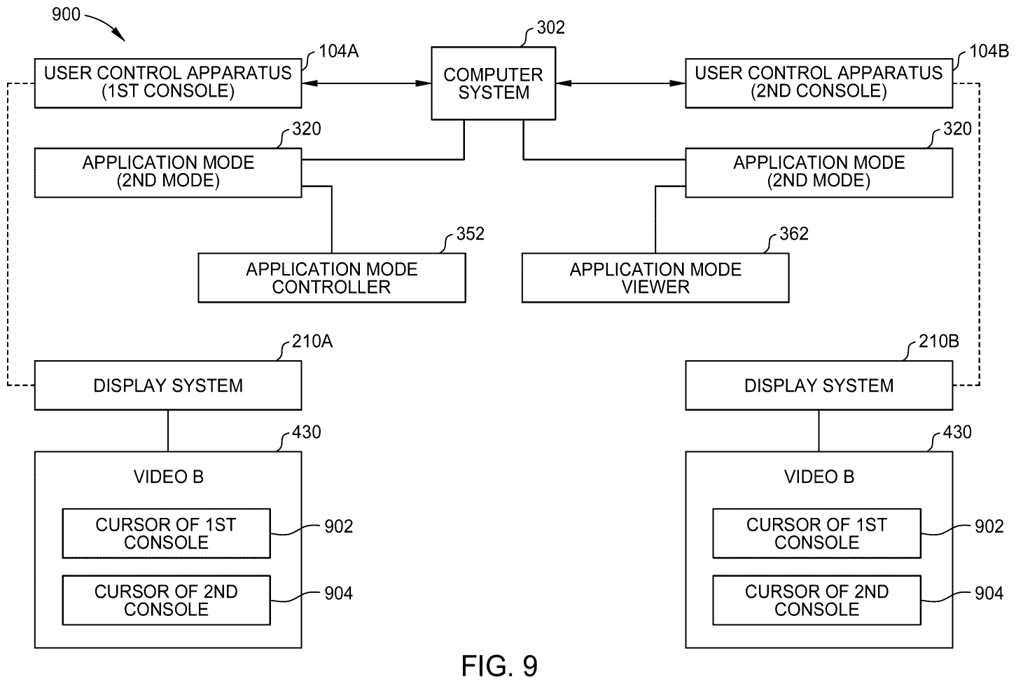

FIG. 9 illustrates an example operation 900 performed by the medical system. Generally, the computer system 302 performs the operation 900 to allow the first operator of the first console 104A to view the cursor of the second operator of the second console 104B, and vice versa.

The operation 900 may occur when both consoles 104A and 104B are in the application mode 320 and the computer system 302 implements a shared application space (e.g., during the operation 700B shown in FIG. 7B). When the first console 104A is in the application mode 320, the first operator of the first console 104A selects an application from the digital menu of applications 432 and launches an application 332. When the second console 104B subsequently transitions to the application mode 320, the computer system 302 communicates a video showing the application 332 to the second console 104B. The computer system 302 and the consoles 104A and 104B may implement a cursor (e.g., a cursor 902 for the first console 104A and a cursor 904 for the second console 104B). The video communicated by the computer system 302 to the console 104B may show the cursor 902 of the first console 104A and the cursor 904 of the second console 104B. As the first operator of the first console 104A moves the cursor 902 to interact with the application 332, the second operator of the second console 104B also sees the movement of the cursor 902. The video communicated by the computer system 302 to the console 104A may also show the cursor 902 of the first console 104A and the cursor 904 of the second console 104B. As the second operator of the second console 104B moves the cursor 904, the first operator of the first console 104A also sees the movement of the cursor 904.

As an example operation, as the first operator of the first console 104A maneuvers the cursor 902, such maneuvering is visible to the second operator of the second console 104B. For example, the computer system 302 allows the second console 104B to view where the first operator of the first console 104A moves the cursor 902. The second operator of the second console 104B may thus follow interactions and manipulations performed by the first operator of the first console 104A and the second operator of the second console 104B may follow the movements of the cursor 902 of the first console 104A. The first operator of the first console 104A becomes the application mode controller because the first operator first entered into the application mode 320. The second operator of the second console 104B becomes the application mode viewer. The computer system 302 prevents the second console 104B from interacting with or changing the presentation in the application mode 320.

The operation 900 may also be performed to show both cursors 902 and 904 on the consoles 104A and 104B when the computer system 302 implements shared application instances (e.g., as in operation 700C shown in FIG. 7C). When both consoles 104A and 104B access the same application 332, the computer system 302 may communicate video to both consoles 104A and 104B that shows both cursors 902 and 904. Movements of one cursor 902 or 904 by one console 104A or 104B may be shown on the other console 104B or 104A.

FIGS. 10A, 10B, and 10C illustrate example operations 1000A, 1000B, and 1000C performed by the medical system. Generally, the computer system 302 performs the operations 1000A, 1000B, and 1000C to determine when the first console 104A transitions from the surgical mode 310 to the application mode 320 and from the application mode 320 back to the surgical mode 310.

In operation 1000A shown in FIG. 10A, the first console 104A starts in the surgical mode 310. The surgical mode 310 may be the default mode for the first console 104A. The first console 104A includes a pedal 1010. When the pedal 1010 of the first console 104A is not pressed or in an inactivated state 1012, the first console 104A is in the surgical mode 310. The computer system 302 communicates the first video 420 (video A) to the first console 104A in the surgical mode 310 to show the surgical site.

In operation 1000B shown in FIG. 10B, the first operator of the first console 104A presses the pedal 1010 such that the pedal 1010 is in the activated state 1014. After pressing the pedal 1010, the first console 104A transitions from the surgical mode 310 to the application mode 320. The application mode 320 remains active as long as the first operator of the first console 104A maintains the pedal 1010 pressed or in the activated state 1014. When the first operator of the first console 104A releases the pedal 1010, the first console 104A returns to the surgical mode 310. The computer system 302 communicates the second video 430 (video B) to the first console 104A to show the menu of applications or a launched application.

In operation 1000C shown in FIG. 10C, when or after the first console 104A transitions to the application mode 320, the computer system 302 communicates, to the second console 104B, a message 1020 indicating a transition of the first console 104A from operating in the surgical mode 310 to operating in the application mode 320. As a result, the second console 104B and the second operator of the second console 104B is informed when the first console 104A transitions from the surgical mode 310 to the application mode 320. The message 1020 may be a visual message or an audible message. The message 1020 may be a notification or indication or alert.

FIG. 11 is a flowchart of an example method 1100 performed by the medical system. For example, the computer system 302 may perform the method 1100. By performing the method 1100, the computer system 302 implements certain features that allow control from two different consoles.

In block 1102, the computer system 302 communicates a first video 420 of a surgical site 422 to the first console 104A and the second console 104B based on determining that both the first console 104A and the second console 104B are operating in a first mode (e.g., the surgical mode 310). In the first mode, the consoles 104A and 104B may remotely operate using a surgical robot in the surgical site 422.

In block 1104, based on determining that the first console 104A transitioned from operating in the first mode to operating in a second mode (e.g., the application mode 320) while the second console 104B operates in the first mode, the computer system 302 communicates, to the first console 104A, a second video 430 of a digital menu of applications 432 while continuing to communicate the first video 420 of the surgical site 422 to the second console 104B. The second console 104B may continue operating the surgical robot, while the first console 104A interacts with the menu of applications 432 or with a launched application 322. In this manner, the computer system 302 allows the consoles 104A and 104B to operate in two different modes concurrently.

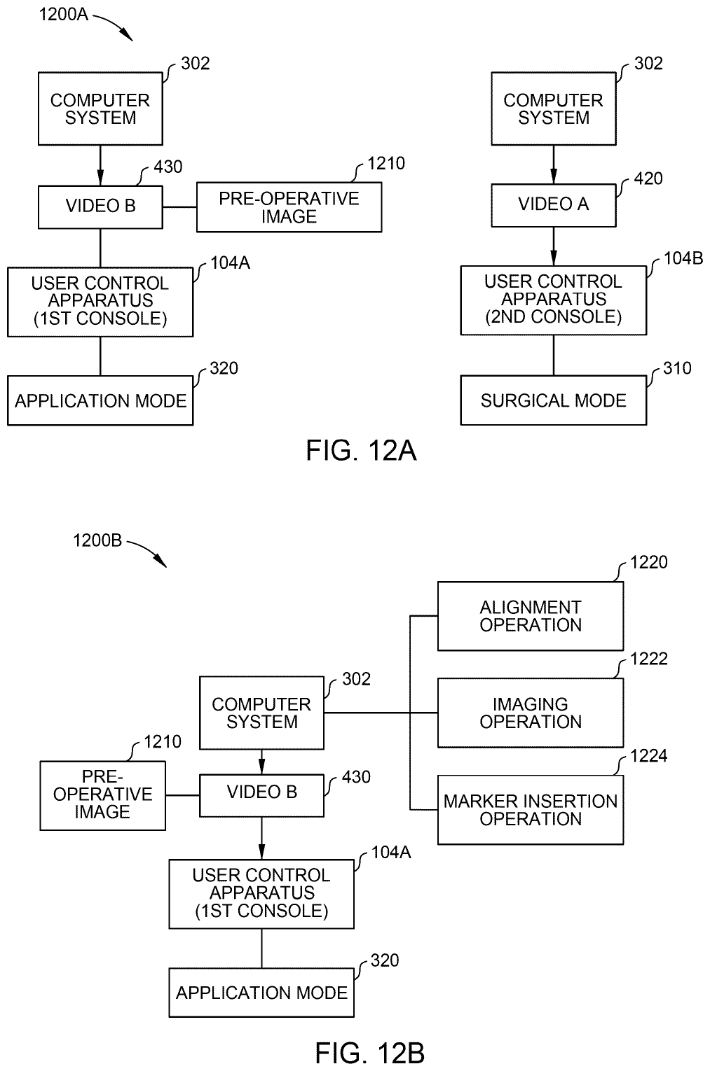

FIGS. 12A and 12B illustrate example operations 1200A and 1200B performed by the medical system. Generally, the computer system 302 performs the operations 1200A and 1200B to show specific applications launched by the first console 104A when the first console 104A transitions from the surgical mode 310 to the application mode 320.

In operation 1200A shown in FIG. 12A, the first console 104A and the second console 104B begin in the surgical mode 310, and then, the first console 104A transitions from the surgical mode 310 to the application mode 320. The computer system 302 then communicates the second video 430 to the first console 104A (e.g., to present the digital menu of applications 432 to the first console 104A or to present a launched application to the first console 104A). The computer system 302 allows the first console 104A to select an application from the multiple available applications. As an example, the selected application may present a pre-operative image 1210. The computer system 302 may thus communicate a pre-operative image 1210 of an object in the surgical site 422 to the first console 104A when the first console 104A operates in the application mode 320 and the second console 104B operates in the surgical mode 310. The first console 104A and the second console 104B may thus operate in different modes. The first console 104A and the second console 104B may thus seamlessly transition between the surgical mode 310 and the application mode 320. As a result, the medical system may provide increased functionality and flexibility. For example, the second operator may perform surgical tasks while the first operator handles digital applications. The first operator handles the digital applications without interrupting the surgical tasks of the second operator.

In one example, the surgical site 422 may be a hernia site showing a hernia. The first console 104A may transition from the surgical mode 310 to the application mode 320 to access the digital menu of applications 432 to select an application to retrieve a pre-operative image 1210 of the hernia. As the first console 104A operates in the application mode 320, the second operator of the second console 104B may remain in the surgical mode 310 and operate on the hernia site. The surgical tasks performed by the second operator of the second console 104B remain uninterrupted by the operations performed by the first operator of the first console 104A in the application mode 320.

In another example, the surgical site 422 is a nephrectomy site navigated by the consoles 104A and 104B to find branches of an artery that feed a mass. The first operator of the first console 104A may transition from the surgical mode 310 to the application mode 320 to access the digital menu of applications 432 to select an application to retrieve a pre-operative image 1210 of the branches of the artery. As a comparison of a location of the branches of the artery with pre-operative scans or a segmented three-dimensional (3D) model is performed by the first console 104A, the second console 104B may remain in the surgical mode 310. The surgical tasks performed by the second operator of the second console 104B remain uninterrupted by the operations performed by the first operator of the first console 104A in the application mode 320.

In yet another example, the first operator of the first console 104A may use contrast agents such as indocyanine green solution (ICG) to visualize cancer cells in the anatomy. ICG dissipates within a few minutes after injection, and the first operator of the first console 104A may want to review a particular area in the anatomy for any residual cancer. The first operator of the first console 104A may draw some markers on the area of the live endoscopic feed that may contain some leftover cancer cells and then ask the second operator of the second console 104B to review the intraoperative video recording. As such, when the first console 104A and the second console 104B are in the application mode 320, the computer system 302 may communicate a message or instruction to the second console 104B to assist in a particular manner. The first operator of the first console 104A is designated as the application mode controller since the first operator first entered into the application mode 320. The second operator of the second console 104B is thus designated as the application mode viewer. The computer system 302 prevents the second console 104B from interacting with or changing the presentation in the application mode 320.

In operation 1200B shown in FIG. 12B, when the first console 104A is in the application mode 320 and selects the application to retrieve a pre-operative image 1210, the computer system 302 communicates the secondo video 430 that includes the pre-operative image 1210 to the first console 104A. The first console 104A may then perform various operations to interact with or manipulate the pre-operative image 1210. The first console 104A may communicate instructions back to computer system 302 to perform these operations. These operations may not interrupt or interfere with the second console 104B, which remains in the surgical mode 310.

In one example, the first console 104A manipulates the pre-operative image 1210 to align the pre-operative image 1210 with the object in the surgical site 422, which may cause the first console 104A to communicate an instruction to the computer system 302 to perform an alignment operation 1220. The computer system 302 performs the alignment operation 1220 and updates the second video 430 to show the changed alignment.

In another example, the first console 104A manipulates the pre-operative image 1210 to show a portion of the object appearing in the video of the surgical site 422, which may cause the first console 104A to communicate an instruction to the computer system 302 to perform an imaging operation 1222. The computer system 302 performs the imaging operation 1222 and updates the second video 430 to show the portion of the object.

In yet another example, the first console 104A manipulates the pre-operative image 1210 to insert markers on the object in the surgical site 422 shown in the pre-operative image 1210, which may cause the first console 104A to communicate an instruction to the computer system 302 to perform a marker insertion operation 1224. The computer system 302 performs the marker insertion operation 1224 and updates the second video 430 to show the inserted markers.

In summary, the present disclosure describes a computer system that allows control from two different consoles. Certain complex surgical procedures are better handled by having a first operator and a second operator work in tandem. For these procedures, more than two instruments, in addition to the camera, are attached to the surgical robotic system. The first operator may handle a first set of instruments (and digital applications) and the second operator may handle a second set of instruments (and digital applications). Each console in the surgical robotic system has two modes of operation. The first mode of operation is a surgical mode in which the operators may control the instruments and the camera through the robotic system. The second mode of operation is an application mode (which may also be referred to as a digital mode) in which the operators may navigate digital menus presented on the console and interact with applications presented on the console. In existing systems, the consoles are restricted to operating in the same mode, which limits functionality and flexibility for the operators. For example, when one console enters the application mode, the other console is also forced into the application mode. However, the present disclosure describes a robotic surgical system in which the consoles may be operated in different modes concurrently. For example, one console may operate in the surgical mode while the other console operates in the application mode. The first operator of the first console and the second operator of the second console may seamlessly transition between the surgical mode and the application mode. As a result, the system provides increased functionality and flexibility. For example, the first operator may perform surgical tasks while the second operator handles digital applications. The second operator handles the digital applications without interrupting the surgical tasks of the first operator.

This description and the accompanying drawings that illustrate aspects, embodiments, or modules should not be taken as limiting. Various mechanical, compositional, structural, electrical, and operational changes may be made without departing from the spirit and scope of this description and the claims. In some instances, well-known circuits, structures, or techniques have not been shown or described in detail in order not to obscure other features. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to be limiting. For example, spatially relative terms-such as “beneath”, “below”, “lower”, “above”, “upper”, “proximal”, “distal”, and the like may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, or module may, whenever practical, be included in other embodiments, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, or application may be incorporated into other embodiments, or aspects unless specifically described otherwise, unless the one or more elements would make an embodiment or embodiments non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted devices and elements in terms of their state in three-dimensional space. As used herein, the term “position” refers to the location of an element or a portion of an element in a three-dimensional space (e.g., three degrees of translational freedom along Cartesian x-, y-, and z-coordinates). As used herein, the term “orientation” refers to the rotational placement of an element or a portion of an element (three degrees of rotational freedom—e.g., roll, pitch, and yaw). As used herein, the term “shape” refers to a set positions or orientations measured along an element. As used herein, and for a device with repositionable arms, the term “proximal” refers to a direction toward the base of the computer-assisted device along its kinematic chain and “distal” refers to a direction away from the base along the kinematic chain.

Aspects of this disclosure are described in reference to computer-assisted systems and devices, which may include systems and devices that are teleoperated, remote-controlled, autonomous, semiautonomous, robotic, and/or the like. Further, aspects of this disclosure are described in terms of an embodiment using a medical system, such as the DA VINCI SURGICAL SYSTEM or ION SYSTEM commercialized by Intuitive Surgical, Inc. of Sunnyvale, California. Knowledgeable persons will understand, however, that aspects disclosed herein may be embodied and implemented in various ways, including robotic and, if applicable, non-robotic embodiments. Techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the disclosure should be limited only by the following claims, and it is appropriate that the claims be construed broadly and, in a manner, consistent with the scope of the embodiments disclosed herein.

What is claimed is:

1. A system comprising:
a surgical robot;
a first console;
a second console; and
a computer system comprising:
a memory; and
a controller communicatively coupled to the memory, the controller configured to:
communicate, to the first console and the second console, video of a surgical site for the surgical robot based on determining that both the first console and the second console are operating in a first mode; and
based on determining that the first console transitioned from operating in the first mode to operating in a second mode while the second console operates in the first mode, communicate, to the first console, video of a digital menu of applications while continuing to communicate video of the surgical site to the second console.

2. The system of claim 1, wherein the controller is further configured to communicate an instruction from the second console to the surgical robot to operate the surgical robot when the second console is operating in the first mode.

3. The system of claim 1, wherein the controller is further configured to navigate the digital menu based on an instruction from the first console when the first console is operating in the second mode.

4. The system of claim 1, wherein the controller is further configured to:
launch an application from the digital menu based on an instruction from the first console; and
communicate video of the launched application to the first console.

5. The system of claim 4, wherein the controller is further configured to transition the launched application from a first state to a second state based on an instruction from the first console.

6. The system of claim 5, wherein the controller is further configured to:
communicate, to the first console, video of the surgical site based on determining that the first console transitioned from operating in the second mode to operating in the first mode after the launched application has transitioned to the second state; and then
communicate, to the first console, video of the launched application in the second state based on determining that the first console transitioned from operating in the first mode to operating in the second mode.

7. The system of claim 6, wherein the controller is further configured to communicate, to the second console, video of the launched application in the second state based on determining that the second console transitioned from operating in the first mode to operating in the second mode after the launched application has transitioned to the second state.

8. The system of claim 4, wherein the controller is further configured to communicate, to the second console, the video of the launched application based on determining that the second console transitioned from operating in the first mode to operating in the second mode while the first console is operating in the second mode.

9. The system of claim 8, wherein the controller is further configured to prevent the second console from changing a state of the launched application while the first console and the second console operate in the second mode.

10. The system of claim 9, wherein the controller is further configured to transition the launched application from a first state to a second state based on an instruction from the second console after the first console transitions from operating in the second mode to operating in the first mode.

11. The system of claim 10, wherein the controller is further configured to communicate, to the second console, video of the digital menu based on determining that the second console transitioned from operating in the first mode to operating in the second mode while communicating video of the launched application to the first console.

12. The system of claim 11, wherein the controller is further configured to communicate, to the second console, the video of the launched application based on receiving, from the second console, an instruction selecting the launched application from the digital menu.

13. The system of claim 8, wherein the video of the launched application communicated to the second console shows a cursor of the first console and a cursor of the second console.

14. The system of claim 1, wherein the first console comprises a pedal, and wherein determining that the first console transitioned from operating in the first mode to operating in the second mode comprises determining that the pedal was pressed.

15. The system of claim 1, wherein the controller is further configured to communicate, to the second console, a message indicating that the first console has transitioned from operating in the first mode to operating in the second mode.

16. The system of claim 1, wherein the controller is further configured to communicate a pre-operative image of an object in the surgical site to the first console when the first console is operating in the second mode and the second console is operating in the first mode.

17. The system of claim 16, wherein the controller is further configured to receive, from the first console, an instruction to manipulate the pre-operative image to align the pre-operative image with the object in the surgical site when the first console is operating in the second mode and the second console is operating in the first mode.

18. The system of claim 16, wherein the controller is further configured to receive, from the first console, an instruction to manipulate the pre-operative image to show a portion of the object appearing in the video of the surgical site when the first console is operating in the second mode and the second console is operating in the first mode.

19. The system of claim 16, wherein the controller is further configured to receive, from the first console, an instruction to manipulate the pre-operative image to insert markers on the object in the surgical site shown in the pre-operative image when the first console is operating in the second mode and the second console is operating in the first mode.

20. A method comprising:
communicating, by a controller to a first console and a second console, video of a surgical site for a surgical robot based on determining that both the first console and the second console are operating in a first mode; and
based on determining that the first console transitioned from operating in the first mode to operating in a second mode while the second console operates in the first mode, communicate, to the first console, video of a digital menu of applications while continuing to communicate video of the surgical site to the second console.
